(19) United States
(12) Patent Application Publication
(10) Pub. No.: US 2005/0209638 A1
(43) Pub. Date: Sep. 22, 2005

(54) Anvil introducer

(76) Inventor: Jon A. Mandelbaum, Indianapolis, IN (US)

Correspondence Address: RANDALL J. KNUTH P.C., 4921 DESOTO DRIVE, FORT WAYNE, IN, 46815, US

(21) Appl. No.: 11/051,579

(22) Filed: Feb. 4, 2005

Related U.S. Application Data
(60) Provisional application No. 60/542,693, filed on Feb. 6, 2004.

(51) Int. Cl. A61B 17/08

(52) U.S. Cl. 606/219

(57) ABSTRACT

The anvil introducer mechanism facilitates the laparoscopic introduction of an anvil, whether for use with a circular stapler (e.g., Ethicon™ stapler) or for use with an Autosuture EEA device, into the abdominal cavity and into the lumen of the stomach or intestine and the placement of such an anvil where desired by the operator. An advantage is that the anvil introduction mechanism greatly eases the passing of an anvil through an abdominal wall and into a particular location in the stomach or intestine. Furthermore, such an introduction mechanism is amenable to use in a laparoscopic procedure and can be readily introduced to a body through a trocar.

### CROSS REFERENCE TO OTHER APPLICATIONS

This application claims priority to U.S. Provisional Patent application 60/542,693, filed Feb. 6, 2003, and which is hereby explicitly incorporated by reference.

### BACKGROUND OF THE INVENTION

1. Field of the Invention

The present invention relates to an anvil introduction mechanism for use with a surgical stapler, and more particular, for use with a circular stapler in stapling portions of the stomach and/or intestines.

2. Description of the Related Art

Laparoscopy is now being used as a less intrusive means of performing bariatric and other surgery such as gastric bypass and of executing colon resections. A key step in laparoscopy and even in open gastrointestinal surgery has been creating successful tissue connections/closures.

Tissue connections (e.g., stomach to intestine; intestine to intestine) are typically often made using a circular stapler in conjunction with an anvil. Such an anvil has a base portion and a projecting portion. The base portion remains behind a first layer of tissue to be connected for later helping bend a staple during stapling to create a connection with that first layer. Meanwhile, the projecting portion is inserted into and through that same first layer of tissue. This projection can then be targeted with the circular stapler to facilitate connection of a second tissue layer to said first layer.

Two different types of generally anvils are used. One is an Ethicon™ anvil available from Ethicon, Inc. of Somerville, N.J., with a female projecting portion and the other is an Autosuture™ anvil available from U.S. Surgical of Norwalk, Conn., having a male projecting portion. This female or male projecting portion is able to connect with a mating portion associated with a selected circular stapler or other connector system, for example Autosuture EEA device. By correctly orienting and connecting the anvil and stapler together, appropriate stapling of tissue segments can be ensured.

Some difficulty has been had in locating, positioning, and controlling the anvil during the stapling procedure. One bariatric procedure has involved introducing the anvil via the esophagus to the appropriate location. This method of introduction has proven to be difficult, especially in achieving the most preferred placement of the anvil. What is needed is an apparatus or device which better facilitates the preferred placement of anvils and which could be used as part of a laparoscopic process and procedure.

### SUMMARY OF THE INVENTION

The anvil introducer mechanism of the present invention facilitates the laparoscopic introduction of an anvil, whether for use with a circular stapler (e.g., Ethicon™ stapler) or for use with an Autosuture™ EEA device, into the abdominal cavity and into the lumen of the stomach or intestine and the placement of such an anvil where desired by the operator.

An advantage of the present invention is that the anvil introduction mechanism greatly eases the passing of an anvil through an abdominal wall and into a particular location in the stomach or intestine. Furthermore, such an introduction mechanism is amenable to use in a laparoscopic procedure and can be readily introduced through a trocar.

Another advantage of the present invention is that the anvil introduction mechanism employs a controllable, flexible arm to allow a high degree of maneuverability.

Yet another advantage of the present invention is that any various handle mechanisms which provide for appropriate motion control of the arm and which provide for the controlled detachment/release of an anvil carried thereby may potentially be employed. This ability to potentially employ any of a variety of appropriate handle mechanisms would allow surgeons to choose a handle mechanism with which they feel most comfortable and assured in controlling.

An additional advantage of the present invention is that any of a variety of control/release anvil holding mechanism may potentially be used. Such a holding mechanism may be for example, magnetic, suction-based, mechanically gripping, and/or frangible in nature.

A further advantage of the present invention is that, while being especially useful for laparoscopic procedures such as gastric bypass, colon resections, and gastronomy, the use thereof can be readily extended to any surgical procedure in which a circular stapler or an autosuture mechanism might be employed.

Another advantage of the present invention is that the anvil introducer permits precise location in an anvil in a desired location. The new anvil introducer is operable with both types of anvils (male and female).

Yet another advantage of the present invention is that it can potentially be made to be inexpensive enough so that at least portions such as the arm and holding mechanisms can be made to be disposable so as to avoid the potential for transmitting foreign material from one patient to another via the anvil introduction mechanism.

### BRIEF DESCRIPTION OF THE DRAWINGS

The above-mentioned and other features and advantages of this invention, and the manner of attaining them, will become more apparent and the invention will be better understood by reference to the following description of the embodiments of the invention taken in conjunction with the accompanying drawings, wherein:

FIG. 1 is a side view of a flexible anvil introduction mechanism of the first embodiment of the present invention;

FIG. 2 is a side view of the flexible anvil introduction mechanism shown in FIG. 1, with the flex arm moved into an angular/flexed position;

FIG. 3 is a side view of a second embodiment of an anvil introduction mechanism of the present invention;

FIG. 4 is a side view of a third embodiment of an anvil introduction mechanism of the present invention;

FIG. 5 is a diagramic side view of a prior art Ethicon™ female anvil;

FIG. 6 is a diagramic side view of a prior art Autosuture™ anvil; and

FIG. 7 is a schematic side view of a laparoscopic, bariatric surgery procedure employing the anvil introduction mechanism of FIGS. 1 and 2.

Corresponding reference characters indicate corresponding parts throughout the several views. The exemplifications set out herein illustrates at least one preferred embodiment of the invention, in one form, and such exemplifications are not to be construed as limiting the scope of the invention in any manner.

### DETAILED DESCRIPTION OF THE INVENTION

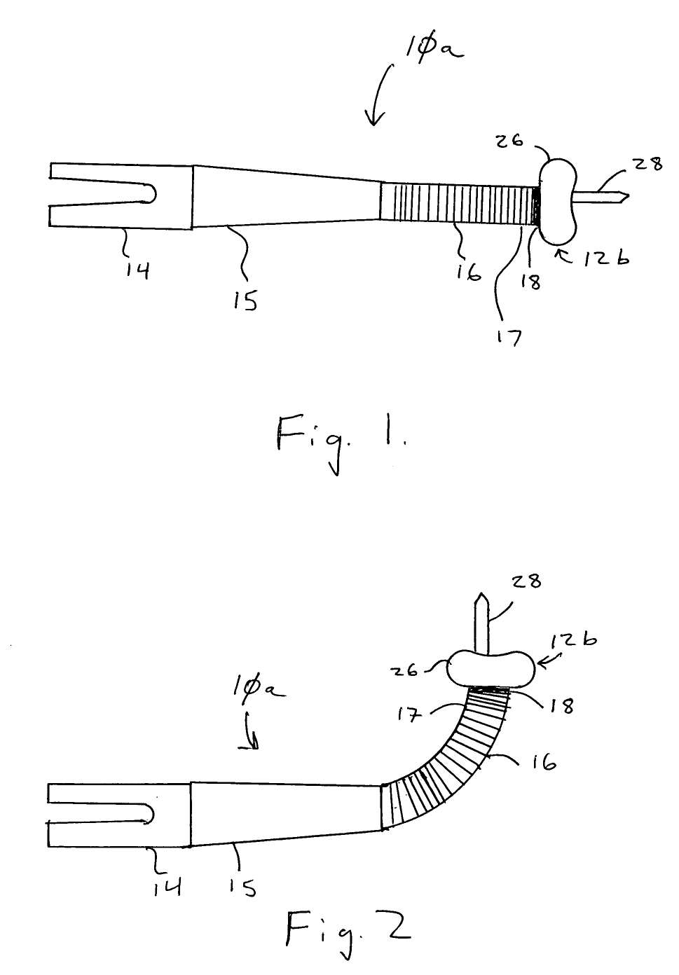

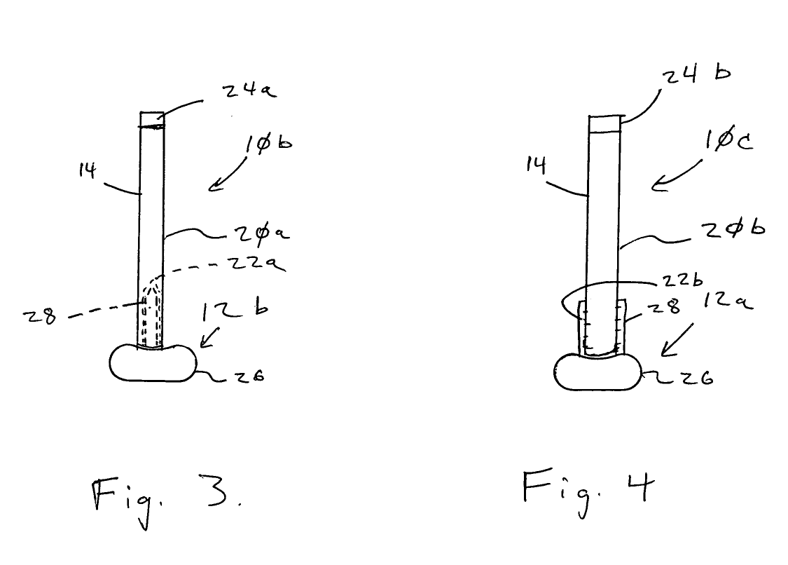

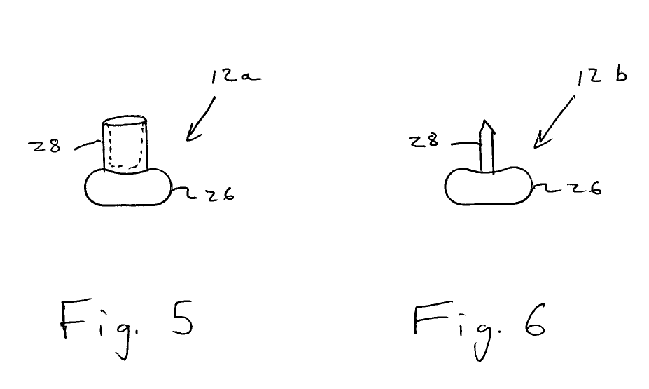

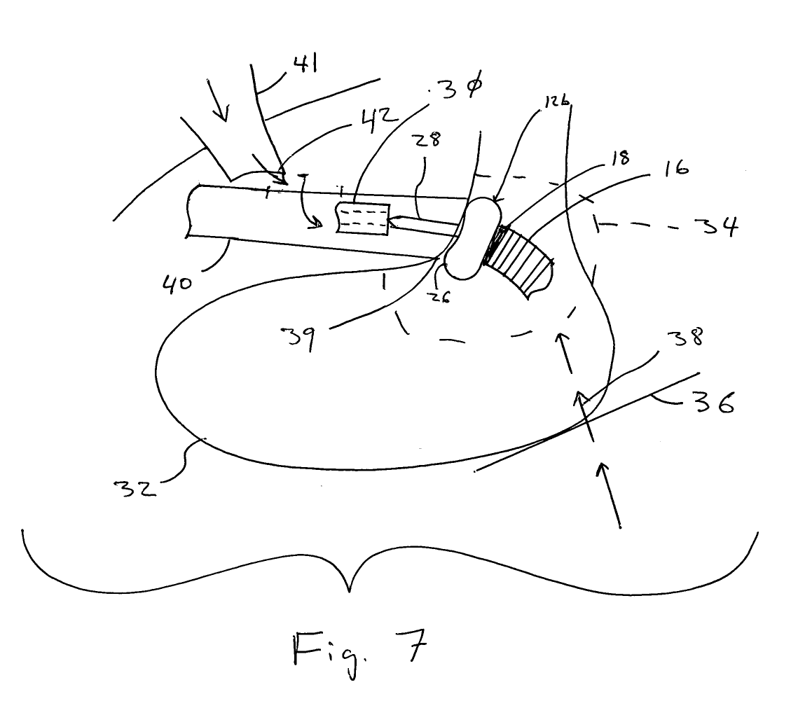

FIGS. 14 illustrate three different embodiments of an anvil introduction mechanism, generally 10, more specifically 10a-10c for introducing an anvil, generally 12, more specifically either a female (e.g., Ethicon™) anvil 12a or a male (e.g., Autosuture™) anvil 12b into an appropriate location within the body and most likely, to a location within the gastrointestinal tract. FIGS. 1 and 2 illustrate a flexible arm anvil introduction mechanism 10a. Meanwhile FIGS. 3 and 4 illustrate a second (10b) and third (10c) embodiment, respectively, of an anvil introduction mechanism to locate the anvil 12.

In the preferred form of the invention (FIGS. 1-2), flexible arm anvil introduction mechanism 10a includes a control handle 14, a flex arm 16, and a releasable attachment mechanism 18.

Control handle 14 is configured for providing an appropriate motion control of the flex arm 16 releasable attachment mechanism 18 combination and for the controllable release of anvil 12a, 12b once suitably placed (if necessary). Any of a variety of particular handle mechanisms which are able to provide for one or both of these functions may be employed, thereby allowing surgeons to choose a handle mechanism 14 which with they feel most comfortable and assured in using. Such handle mechanisms may include flex tube controllers, or even simple flexible wire or bendable constructs (for example large gauge wire), that would permit operator control of the placement of anvil 12. Some handles and embodiments of invention may include no remote releasement mechanism from anvil 12.

The flex arm/attachment mechanism combination, along with the anvil 12a, 12b carried thereby, are together sized to fit through a trocar as typically employed in a laparoscopic procedure. Flex arm 16 is configured for both its flexibility and its controllability, thereby allowing an anvil 12a, 12b to be both easily and precisely guided into place. As per the design of this first embodiment (FIGS. 1-2), the flex arm actually guides/pushes anvil 12a, 12b from behind and into the desired location. It is to be understood that flex arm 16 can be produced so as to have any of a variety of desired lengths and potentially be controllably expandable/contractible along its length so as to facilitate its travel to the desired location and back out of the body. Flex arm 16 includes a first arm end 15 and second arm end 17. Releaseable attachment mechanism is located and associated with second arm end 17, while handle 14 is located and associated with first arm end 15.

Releasable attachment mechanism 18 is configured to retain anvil 12a, 12b upon flex arm 16 up until the point at which it is desired to release it after having been placed into the desired location (e.g., the position within of the stomach and/or intestine where stapling is desired). It is important that the releasable attachment mechanism 18 be reliable in this manner, as it is not desired be allowed for an anvil 12 to be accidentally released prior to reaching its desired location. It is understood that any one of a variety of potential means may be employed to achieve the desired control-release by the anvil holding mechanism. Such means may be, for example, magnetic, suction-based, mechanically gripping, interference fit, push-pull rod, frangible, or snap-locking in nature. Since there is a potential for an anvil 12a, 12b to be inadvertently released prior to being located in the appropriate spot, it may prove useful to choose a holding means which may allow for a grip to be easily re-established with an anvil 12a, 12b. A magnetic means would be preferable in this regard, and is one aspect of the present invention.

Second and third embodiments 10b, 10c of the anvil introduction mechanism of the present invention are very similar in nature in that both are configured for placing an anvil 12a, 12b into a desired location, with anvil 12a, 12b being released once such a location is reached. The second and third embodiments differ from each other in that the second embodiment is configured for carrying a male anvil 12b (FIG. 3), while the third embodiment 10c is designed for carrying a female anvil 12a (FIG. 4). The second embodiment of the anvil introduction mechanism 10b includes a carrying rod 20a, a receiver portion 22a, and a mechanical release control 24a. In a similar fashion, third embodiment 10c (FIG. 4) of the anvil introduction mechanism includes a carrying rod 20b, a male receiver portion 22b, and a mechanical release control 24b. Male receiver portion 22b interfits within the female portion of anvil 12a.

Carrying rod 20a, 20b provides a mechanism by which the particular anvil introduction mechanism 10b, 10c pushed through a trocar opening, into and through a desired location in a wall of the stomach and/or intestine and, after release of anvil 12a, 12b, eventually therefrom on out through a particular trocar opening. At one distal end of a carrying rod 20a, 20b is a receiver portion 22a, 22b, while at the opposing distal end has a mechanical release control 24a, 24b, respectively.

Receiver portion 22a, 22b and mechanical release control 24a, 24b are able to work together to ensure that the anvil 12a, 12b is retained upon receiver portion 22a, 22b until the desired release location is reached. Receiver portion 22a is female in nature so that it may carry a male anvil 12b thereupon, while receiver portion 22b is a male connector in order to transport a female anvil 12a therewith. In relation to the anvil introduction mechanism of the second and third embodiment, it is considered desirable for the anvil 12a, 12b initially to form a snap fit in relation to receiver portion 22a, 22b. The receiver portion 22a, 22b is configured to have the means to releasably grip an anvil 12a, 12b. Any of a variety of gripping/holding means may be used for this purpose, but it is advantageously conceived that the particular means for holding may allow for the extrusion of the anvil therefrom upon arriving at the appropriate location. It is contemplated that the mechanical release control 24a, 24b may, for example, take the form of a push button, push bar, or a twist-control device which will achieve the desired controllable release of anvil 12a, 12b from receiver portion 22a, 22b.

Each anvil 12a, 12b includes an anvil base 26 and an anvil projection 28. This anvil base 26 facilitates the bending of a staple inserted via a circular stapler schematically in FIG. 7 as part 30. Anvil base 26 is wide enough (typically 25 mm in diameter) so as to both facilitate the appropriate bending of a given staple (not shown) and to ensure that the anvil will not be drawn through a particular opening in the wall of the intestine and/or stomach of where it is located and stapling is desired. Anvil projection 28 is configured so as to be able to be inserted through the wall of the stomach and/or intestine and to in turn then act as a locating means for the placement of the circular stapler so as to ensure the desired staple location is met. Such an anvil projection 28 is typically 2 inches in length.

The anvil locator of the present invention may be contructed of autoclavible materials and metals, or may be constructed primarily of plastic, as a single use and dispose of, style instrument.

An example of the invention, is shown in a bariatric surgery example shown in FIG. 7. The first step of this procedure is to, within stomach 32, create a pouch 34. Next, stomach 32 has a stomach cut 36 formed therein (either with a blade or tocar) to permit insertion of anvil 12a, 12b with flexible arm anvil introduction mechanism 10a. A tocar (not shown) may be used to insert anvil introduction mechanism. Introduction mechanism 10a and anvil 12a, 12b is directed along an anvil path 38 to an appropriate anvil location 39 within the wall of stomach 32. Having located anvil 12a, 12b, small intestine 40 is provided with a small intestine cut 42 through which a circular stapler 30 may be introduced. Such a small intestine cut 42 is made with the appropriate instrumentation (not shown) provided through a trocar 41. Given the tight fit of the circular stapler 30, its introduction requires the removal of trocar 40. After having provided for appropriate insertion of circular stapler 30, small intestine 40 can then be located on pouch 34. Upon doing so, circular stapler 30 is then located and connected to anvil 12a, 12b, and small intestine 40 is then attached to pouch 34 via stapling. After stapling both the stapler, anvil 12 and anvil introducer may be removed from the location.

While this invention has been described as having a preferred design, the present invention can be further modified within the spirit and scope of this disclosure and additionally be used on other body parts. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An anvil introduction mechanism configured for use in a laparoscopic procedure, said anvil introduction mechanism comprising:
a controllable arm mechanism, said arm mechanism having a first arm end and a second arm end;
an attachment mechanism associated with said second arm end for holding an anvil;
a handle control device associated with said first arm end for controlling said anvil introduction mechanism.

2. The anvil introduction mechanism of claim 1 in which said attachment mechanism is releasable, said releasable attachment mechanism configured for releasably holding an anvil member at said second arm end; and

3. The anvil introduction mechanism of claim 1 in which said handle control device is configured for selectively moving said arm mechanism and for thereby locating the anvil carried at said second arm end,

4. The anvil introduction mechanism of claim 3 in which said said handle control device being further configured for selectably activating said releasable attachment mechanism to thereby release the anvil.

5. The anvil introduction mechanism of claim 1 in which said attachment mechanism utilizes a snap-lock to connect to an anvil.

6. The anvil introduction mechanism of claim 1 in which said attachment mechanism utilizes a frangible connection to connect to an anvil.

7. The anvil introduction mechanism of claim 1 in which said attachment mechanism utilizes an interference fit to connect to an anvil.

8. The anvil introduction mechanism of claim 1 in which said arm is flexible.
